(19) United States
(12) Patent Application Publication
(10) Pub. No.: US 2015/0080744 A1
(43) Pub. Date: Mar. 19, 2015

(54) METHOD AND SYSTEM FOR ASSESSING PRETERM BIRTH AND OTHER PATHOLOGIES

(71) Applicants: The Johns Hopkins University, Baltimore, MD (US); The University of Texas Southwestern Medical Center, Dallas, TX (US)

(72) Inventors: Xingde Li, Ellicott City, MD (US); Yuying Zhang, Ann Arbor, MI (US); Wenxuan Liang, Baltimore, MD (US); Mala Mahendroo, Arlington, TX (US); Kate Luby-Phelps, Arlington, TX (US); Meredith Akins, Fort Smith, AR (US)

(21) Appl. No.: 14/454,125

(22) Filed: Aug. 7, 2014

Related U.S. Application Data
(60) Provisional application No. 61/863,022, filed on Aug. 7, 2013.

(51) Int. Cl. A61B 5/00; A61B 1/06; A61B 5/107; A61B 1/00; A61B 1/07; A61B 1/303

(52) U.S. Cl. CPC A61B 5/435; A61B 1/07; A61B 1/303; A61B 5/0084; A61B 5/1076; A61B 5/1079; A61B 5/7275; A61B 1/00009; A61B 1/0669; A61B 5/0062; A61B 5/7282
USPC 600/478

(57) ABSTRACT

The present invention is directed to an all-fiber-optic scanning endomicroscope capable of high-resolution second harmonic generation (SHG) imaging of biological tissues. The endomicroscope has an overall 2.0 mm diameter and consists of a single customized double-clad fiber, a compact rapid two-dimensional beam scanner, and a miniature compound objective lens for excitation beam delivery, scanning, focusing, and efficient SHG signal collection. Endomicroscopic SHG images of murine cervical tissue sections at different stages of normal pregnancy reveal progressive, quantifiable changes in cervical collagen morphology with resolution similar to that of bench-top SHG microscopy. A device according to an embodiment of the present invention can also be used to assess other pathologies, such as cancer. fibrosis, and inflammation. The present invention allows for diagnosis, monitoring of the effect of therapeutics, and surgical or interventional guidance. The present invention enables visualization of histology in-vivo, in the patient, and is label-free.

### CROSS REFERENCE TO RELATED APPLICATIONS

This application claims the benefit of U.S. Provisional Patent Application No. 61/863,022 filed on Aug. 7, 2013, which is incorporated by reference, herein, in its entirety.

### GOVERNMENT SUPPORT

This invention was made with government support under grant no. CA153023 awarded by the National Institutes of Health. The government has certain rights in the invention.

### FIELD OF THE INVENTION

The present invention relates generally to medical imaging. More particularly, the present invention relates to a method and system for assessing preterm birth and other pathologies using medical imaging.

### BACKGROUND OF THE INVENTION

The cervix is a connective tissue-rich structure just caudal to the uterus in female mammals. Appropriate remodeling of the cervix during gestation is an essential component of the birth process. The cervix must remain closed during pregnancy to maintain the fetus in the womb, and then open sufficiently to allow passage of the fetus at term. This shift in responsibility requires a massive rearrangement of the cervical connective tissue, in particular fibrillar collagen. Collagen is the main structural protein in the cervix. Animal studies reveal rearrangement of collagen structure is achieved in part by a decline in collagen crosslink formation, a reduction in matricellular proteins that regulate collagen fibrillogenesis, and increased synthesis of the matrix disorganizing molecule, hyaluronan. Evidence to support a conservation of these processes in cervical remodeling in women is mounting. Abnormal or inappropriately timed cervical remodeling can lead to premature birth.

Preterm birth (PTB), which accounts for 12.7% of all births in the United States, is the second leading cause of infant mortality and often leads to serious morbidity in surviving infants. Despite considerable research, the cause of PTB in 50% of cases remains elusive, and diagnostic methods to detect women at risk of PTB are limited. Because clinical and animal studies suggest that cervical changes precede the onset of uterine contractility in both term and PTB, premature cervical changes could be indicative of impending PTB. The progressive structural changes in fibrillar collagens are directly related to cervical rigidity and thus potentially can serve as a diagnostic indicator for women at risk for PTB.

Second harmonic generation (SHG) microscopy is the most effective method for direct noninvasive imaging of collagen type I in biological tissues at submicrometer resolution. Collagen I is the most abundant collagen in the cervical matrix. A recent study used SHG microscopy to reveal progressive changes in collagen matrix architecture in mouse cervical tissue sections during normal pregnancy and in one mouse model of PTB. This method was able to detect quantifiable changes in cervical collagen earlier than biomechanical changes can be detected. Quantification of these changes by relatively simple metrics showed the progress of collagen matrix remodeling at various stages of normal pregnancy and revealed unexpected insights regarding the mechanism of one mouse PTB model. Application of this emerging technology in the clinic thus holds the promise for significant improvement in assessment and prediction of the risk for preterm birth.

Translation of cervical SHG microscopy for clinical use in vivo represents a formidable engineering challenge, which requires miniaturization of a bench-top microscope down to a small imaging probe applicable for minimally invasive imaging of the cervix. Fiber optics-based miniature and flexible imaging devices such as endoscopes and catheters have established new landscapes for clinical applications of high-resolution optical imaging technologies, including optical coherence tomography, confocal imaging, and more recently, nonlinear optical imaging. Several critical technological challenges and engineering building blocks for developing a high-performance SHG endomicroscopy technology exist. In addition to the challenges in effective excitation pulse delivery, SHG signal collection and miniature beam scanner, one significant challenge for an SHG imaging endomicroscope is the more restrictive demand for low chromatic aberration in the micro-objective lens, considering the large wavelength separation between the excitation and emission in SHG imaging (which is typically much larger than the separation in two-photon fluorescence imaging).

Accordingly, there is a need in the art for a SHG endomicroscopy probe for use in imaging procedures.

### SUMMARY OF THE INVENTION

The foregoing needs are met, to a great extent, by the present invention which provides a method of assessing preterm birth in vivo including using a fiber-optic scanning second harmonic generation (SHG) endoscopy imaging system. The method includes obtaining SHG endoscopy images of cervical collagen. The method also includes determining collagen fiber morphologies from the SHG endoscopy images.

In accordance with an aspect of the present invention, the collagen fiber morphologies include at least one selected from the group consisting of collagen fiber diameter, volume, porosity, and fiber bending angle. The collagen fiber morphologies are determined using a computing device. Abnormal changes in collagen fiber morphologies are determined in order to determine a risk of pre-term birth. The SHG endoscopy imaging system is used in situ, in vivo, and in real time. The SHG endoscopy system includes a short pulsed laser source, a fiber optic scanning endoscope, light detection unit, and computer control. The SHG endoscopy imaging system can obtain images from the front and from the side.

### BRIEF DESCRIPTION OF THE DRAWINGS

The accompanying drawings provide visual representations, which will be used to more fully describe the representative embodiments disclosed herein and can be used by those skilled in the art to better understand them and their inherent advantages. In these drawings, like reference numerals identify corresponding elements and:

FIG. 1A illustrates a schematic of a distal end of the SHG endomicroscope probe.

FIG. 1B illustrates a photo of a cross section of a single double-clad fiber.

FIG. 1C illustrates a photograph of the distal end of the endomicroscope.

FIG. 1D illustrates a schematic diagram of an entire endomicroscope system setup.

FIGS. 2A and 2B illustrate a two-photon excited fluorescence intensity profile of a 0.1 μm diameter fluorescent bead collected by the endomicroscope along the lateral and axial direction respectively.

FIGS. 3A-3C illustrate exemplary endomicroscope images.

FIGS. 4A-4J illustrate representative images of cervical sections from non-pregnant mice and mice at days 6, 12, 15, and 18 of pregnancy.

FIG. 5A illustrates a graphical view of characteristic fiber size increased progressively from non-pregnant to day 18 of gestation.

FIGS. 5B and 5C illustrate a graphical view of fractional area and mean gray value from non-pregnant to day 18 of gestation.

FIGS. 6A-6H illustrate exemplary photographs of mouse cervix.

### DETAILED DESCRIPTION

The presently disclosed subject matter now will be described more fully hereinafter with reference to the accompanying Drawings, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Drawings. Therefore, it is to be understood that the presently disclosed subject matter is not to be limited to the specific embodiments disclosed and that modifications and other embodiments are intended to be included within the scope of the appended claims.

The present invention is directed to an all-fiber-optic scanning endomicroscope capable of high-resolution second harmonic generation (SHG) imaging of biological tissues. The endomicroscope has an overall 2.0 mm diameter and consists of a single customized double-clad fiber, a compact rapid two-dimensional beam scanner, and a miniature compound objective lens for excitation beam delivery, scanning, focusing, and efficient SHG signal collection. Endomicroscopic SHG images of murine cervical tissue sections at different stages of normal pregnancy reveal progressive, quantifiable changes in cervical collagen morphology with resolution similar to that of bench-top SHG microscopy. SHG endomicroscopic imaging of ex-vivo murine and human cervical tissues through intact epithelium has also been performed and show the applicability of the device as a minimally invasive tool for clinical assessment of abnormal cervical remodeling associated with preterm birth. A device according to an embodiment of the present invention can also be used to assess other pathologies, such as cancer. fibrosis, and inflammation. The present invention allows for diagnosis, monitoring of the effect of therapeutics, and surgical or interventional guidance. The present invention enables visualization of histology in-vivo, in the patient, and is label-free.

As noted, the SHG endomicroscope of the present invention has improved nonlinear signal collection efficiency and resolution. The performance of the SHG endomicroscope is demonstrated for assessing cervical collagen fiber morphology in the mouse cervix ex vivo and morphological changes in SHG images correlated with established histological findings. Quantitative analyses of endomicroscopic images revealed a progressive increase in collagen fiber size and SHG signal intensity during the course of pregnancy as previously shown by bench-top SHG microscopy. Differences in collagen signal were observed very early in pregnancy, allowing the identification of structural aberrations well before changes in biomechanical properties of the tissue could be detected. The promise of the endomicroscope for future in vivo studies was further demonstrated by resolving mouse and human cervical collagen fiber morphologies through the intact epithelium. This work provides proof-of-principle that SHG endomicroscopy has potential as a noninvasive method to assess cervical remodeling status during pregnancy, from which PTB-associated risk can potentially be revealed.

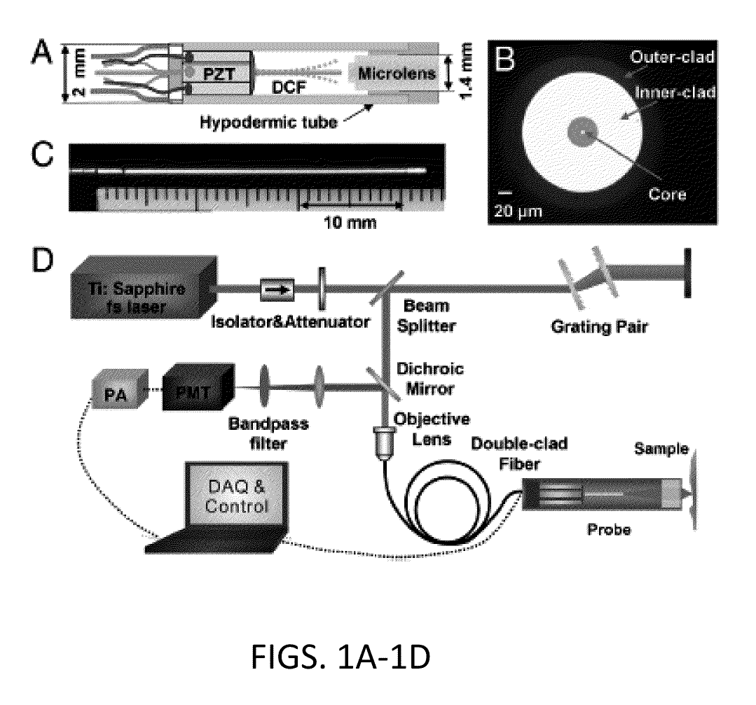

FIG. 1A shows a schematic of the distal end of the SHG endomicroscope probe where a single double-clad fiber (DCF) was used for both femtosecond excitation light delivery to and SHG signal collection from the sample. The use of a single DCF (along with other miniature components as described below) helps reduce the overall probe size. The fundamental working principle has been detailed elsewhere. In essence, the probe consists of three main parts: (i) a four-quadrant piezoelectric (PZT) tube to actuate a fiber cantilever, (ii) a single piece of DCF running through and glued to the end of the PZT tube to serve as a cantilever and perform fast two-dimensional beam scanning, and (iii) a miniature objective lens at the distal end of the probe to focus the excitation beam and collect the SHG signal. In order to develop a high-performance nonlinear optical imaging endomicroscope, several new advancements have been introduced to the basic design of the endoscope. First, a new customized DCF (Coming Inc.) was developed. The DCF has a single-mode core of 5.5-μm diameter and a 0.12 numerical aperture (NA) for delivering excitation femtosecond pulses and a large inner-clad of a 185-μm diameter and 0.3 NA for collecting the SHG signal. A photo of the DCF cross-section is shown in FIG. 1 B. Compared to a commercially available DCF (with an inner clad diameter of 100 μm and an NA of 0.23), the larger inner-clad diameter and higher NA of the customized DCF improves SHG signal collection efficiency by approximately 4.5-fold [i.e., [symbol] 1.85/100 [symbol] 2× [symbol] 0.3/0.23 [symbol] ]. In addition, a miniature compound lens with an NA of 0.8 and a working distance of 200 μm in water was utilized. The microlens has super achromatic performance with negligible focal shift between the excitation (800-900 nm) and the SHG signal (400-450 nm). Ray tracing analysis indicates that the maximum focal shift is less than 20 μm between 890 nm (excitation) and 445 nm (SHG emission), which corresponds to a cone with an end diameter of 4.8 μm for the SHG light when focused back to the DCF facet. That is much smaller than the 185-μm collection diameter of the DCF. The superb achromatic performance of the micro-objective lens further improved the SHG collection efficiency by more than eightfold when experimentally compared to a commonly used gradient index (GRIN) lens.

The DCF cantilever, tubular PZTactuator and microlens were assembled into a hypodermic tube with an overall dimension of 2 mm×32 mm (diameter×length). A photograph of the distal end of the endomicroscope is shown in FIG. 1C. The small form factor of the endomicroscope also enables its potential integration with other medical instruments for various clinical applications in vivo. The schematic of the entire endomicroscopy system setup is shown in FIG. 1D, which includes the femtosecond laser, the dispersion management unit, the scanning endomicroscope, and the SHG signal detection unit. The details about the optical system setup are described in the Methods.

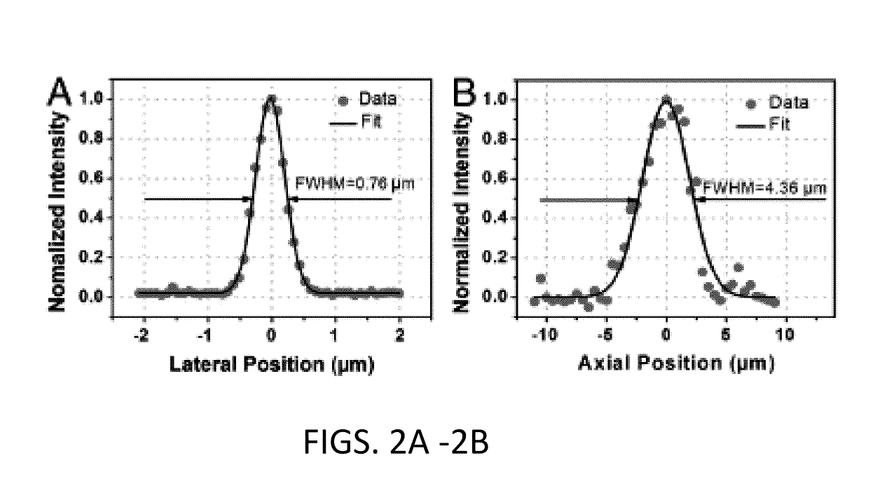

The high-NA microobjective lens in the endomicroscope enabled us to achieve a small focused spot size and improved lateral resolution by almost twofold over previously reported systems. FIGS. 2A and 2C shows the two-photon excited fluorescence intensity profile of a 0.1-μm diameter fluorescent bead collected by the endomicroscope along the lateral (FIG. 2A) and axial (FIG. 2B) direction, respectively. The lateral and axial resolutions of the endomicroscopy system, provided by the full-width-at-half maximum of the Gaussian fit to the respective fluorescence intensity profile, were about 0.76×4.36 μm (lateral×axial), which were close to predicted resolutions of 0.69×6.15 μm. The discrepancy is probably attributable to the potential inaccuracy in moving the imaging beam through the small fluorescent bead, a slight lateral position shift (e.g., off the bead center) during the axial scan, and the potential spherical aberration in the microlens.

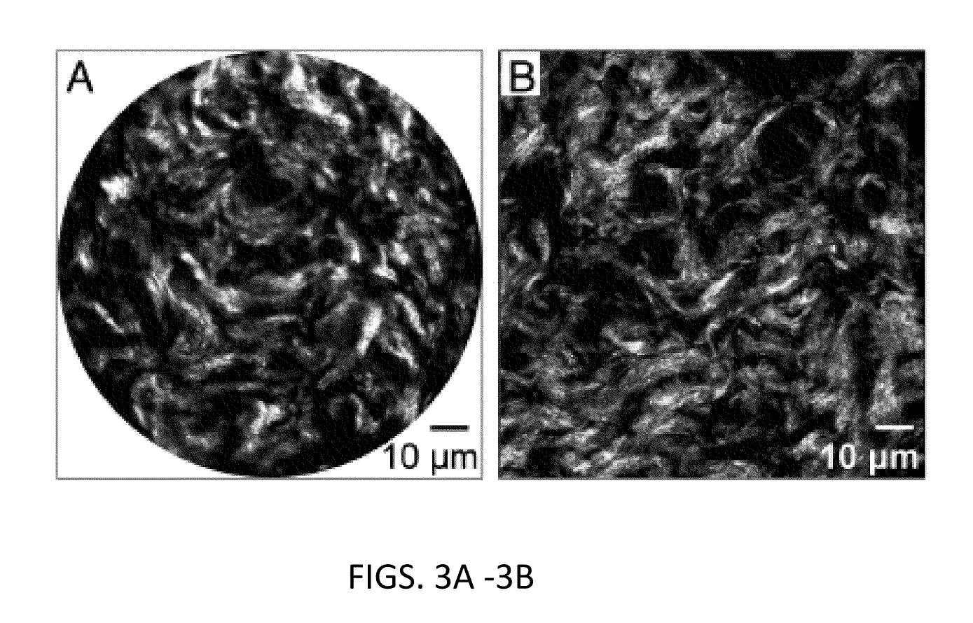

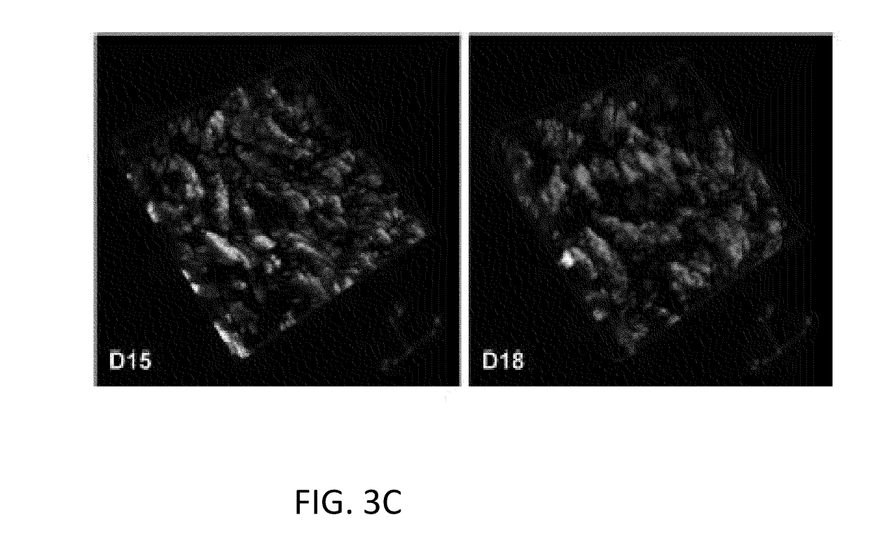

Mice (C57B16/129S) have a gestation time of approximately 19 d with birth occurring early on day 19. Biomechanical measurements of cervical tissue identify a progressive increase in tissue compliance that is measurable by day 12 of pregnancy and reaches its maximum at birth. The accompanying changes in cervical collagen morphology have been well documented in mouse tissue sections using bench-top SHG microscopy. To assess the performance of the SHG endomicroscope, images of frozen sections of cervix from mice at various stages of pregnancy were acquired with the endomicroscope and compared with images of the same tissue section acquired with benchtop SHG microscope. To allow quantitative comparisons, the SHG imaging conditions were kept constant for all samples. The bench-top SHG microscope is equipped with a 20×0.95 NA objective lens. The incident power (and temporal pulse width) on the tissue sample was approximately 40 mW (450 fs) and 15 mW (150 fs) for the endomicroscope and microscope, respectively. The imaging speed was 2.7 frames/second with the endomicroscope and 0.37 frames/second for the microscope. FIG. 3A shows a representative SHG endomicroscopy image of a cervical section from a mouse at day 18 of gestation, where the fibrillar collagen morphologies can be clearly resolved. The endomicroscope offers a field of view (FOV) of about 110 μm in diameter on the sample. FIG. 3B shows the corresponding microscopy SHG image acquired from a similar region with the image size chosen to match the endomicroscope FOV. Considering the differences in the imaging speed, temporal pulse width, incident power, and potential collection efficiency in the two imaging systems, 20 frames and 4 frames were averaged for the endomicroscopy and benchtop benchtop microscopy systems, respectively, to achieve a comparable signal-to-noise ratio. These images show that the endomicroscope offered a sufficient excitation and SHG collection efficiency with a resolution approaching a bench-top microscope and image quality sufficient to resolve the fine details of collagen morphology. Image quality is also sufficient to perform 3D imaging by translating the specimen through the focal plane of the endomicroscope with a precision PZT stage. 3D projections of mouse cervical tissue sections on gestation day 15 and day 18 are shown in FIG. 3C.

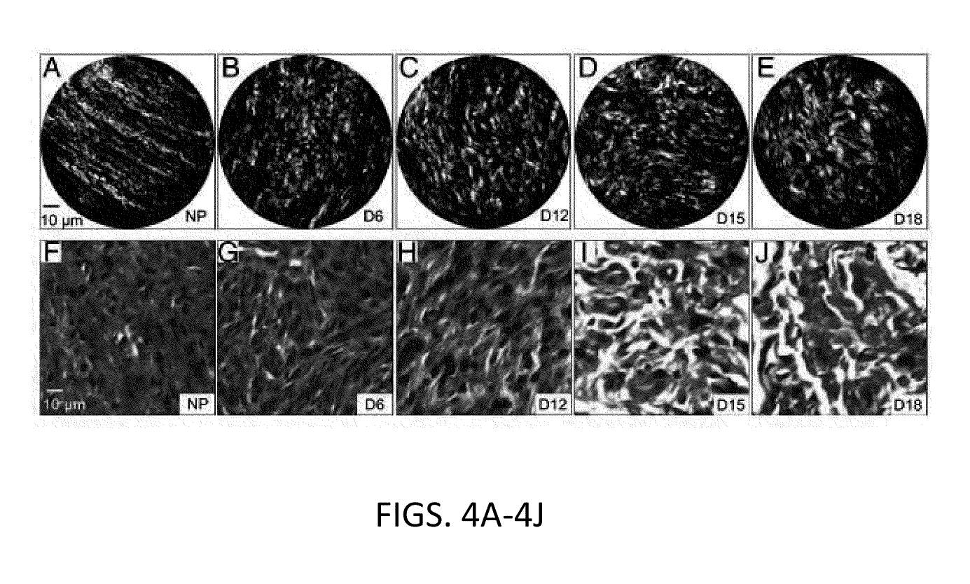

FIGS. 4A-4J show representative images of cervical sections from nonpregnant (NP) mice and mice at days 6, 12, 15, 18 of pregnancy. SHG microscopy has previously shown that there is a progressive increase in SHG intensity from early to late pregnancy and this was also evident in the endomicroscope data. The contrast of the images in FIGS. 4A-E has been adjusted to compensate for intensity differences and optimize visualization of morphological details. The SHG images obtained by endomicroscopy at each stage of pregnancy appeared very similar to those obtained with the bench-top SHG microscope from the same stage of pregnancy, revealing morphological changes of the collagen fiber matrix during pregnancy as reported previously. Collagen fibers were highly aligned, thin, and relatively straight in the NP cervix and in early pregnancy (e.g., at day 6 of gestation). The collagen fibers gradually became more curved and thicker in appearance in the later stages of pregnancy. FIGS. 4A-J show that the progressive changes in morphology of collagen in the SHG images (FIGS. 4A-E) correlate generally with images of trichrome stained cervical tissue sections (FIGS. 4F-J) at each stage of pregnancy and highlights the power of SHG endomicroscopy to reveal fine details of collagen matrix architecture.

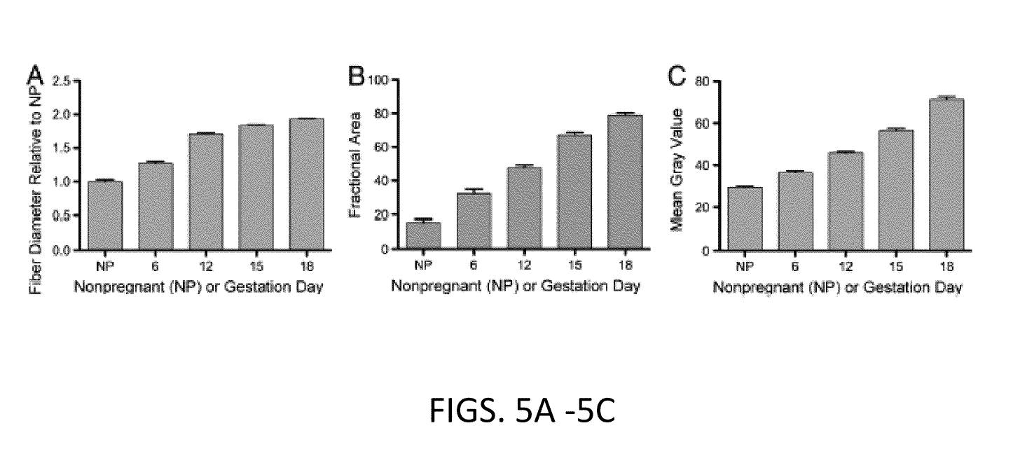

For quantitative analysis of morphology, the images were normalized locally by contrast stretching each image individually to fill an 8-bit grayscale as described in Methods. FIG. 5A shows that characteristic fiber size increased progressively from NP to day 18 of gestation (P<0.0001 at all-time points). In addition, intensity based analyses were also performed to extract quantitative information such as the fractional area and mean gray value (MGV) of the SHG signals. For these intensity-dependent measurements, the images were normalized globally by adjusting all images at all-time points to a fixed grayscale chosen from the minimum and maximum gray values in the aggregate dataset. Different from local normalization, which best reveals the morphological details in each image; the global normalization preserves the relative differences in SHG signal intensity at each time point. As shown in FIGS. 5B and C, the resulting fractional area and MGV of the SHG signal above threshold progressively increased throughout pregnancy (P<0.0001 at all time points), reaching a maximum on day 18. These findings confirm that resolution and quality of the endomicroscopy images are sufficient to detect the quantitative changes previously documented only by bench top SHG microscopy.

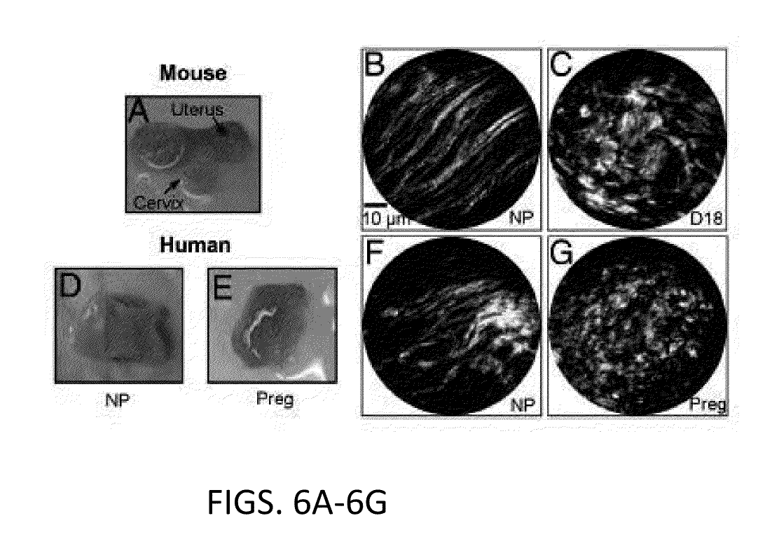

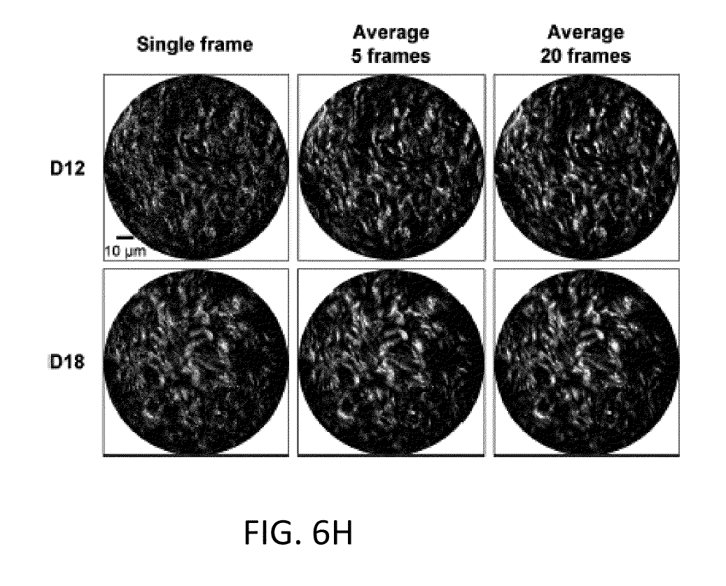

In addition to cervical tissue sections, ex vivo mouse cervical tissues (NP and gestation day 18) with intact epithelium were also used for testing the performance of the SHG endomicroscopy. FIG. 6A shows a photo of an excised NP mouse cervix placed on a wax block. FIGS. 6B and C show representative SHG endomicroscopy images from NP and day 18 mouse cervical tissue with the probe placed on the outside of the exocervix. Although epithelial cell proliferation during pregnancy leads to an increase in the number of epithelial cell layers, total epithelial thickness is not well documented. Based on preliminary measurements from multiple NP and gestation day 18 paraffin sections, the epithelial thickness varies within each time point and ranges from 30 μm to 400 μm thick with an average thickness of about 163 μm. Instead of averaging over 20 frames, only 5 frames of averaging were applied. This reduced the total time to acquire an averaged image from 7.4 s down to 1.85 s. A comparison of image quality based on number of frames averaged is shown in FIG. 6H. The SHG endomicroscopy images from mouse tissues through epithelium show similar quality and morphological features as acquired from mouse sections when comparing FIGS. 4A and E with FIGS. 6B and C. The imaging capability of the endomicroscope was further tested using human cervical NP and term pregnant specimens with a photo shown in FIGS. 6D and E, respectively. The representative SHG endomicroscopy images are illustrated in FIGS. 6F and G. Similar to the mouse cervix, the fine cervical collagen architecture can be clearly observed through the epithelium and the collagen structure clearly changes from long thin collagen fibers in the NP cervix to curved, thicker fibers in term pregnant cervix.

The compact fiber-optic SHG endomicroscope has a resolution and image quality comparable to a bench-top microscope. Compared to previously reported nonlinear optical endomicroscope prototypes, the current model offers a significant improvement in signal collection efficiency (i.e., by more than 30-fold), owing to the use of a specially designed DCF with a large SHG collection area and numerical aperture and the introduction of a super achromatic micro-objective lens. The resolution was also improved by almost threefold compared to a first prototype, benefiting from the high NA of the microobjective lens. The choice of a single DCF configuration for excitation laser delivery, SHG signal collection and beam scanning (along with a tubular piezoelectric actuator), made it possible to engineer a scanning endomicroscope with an exceptionally compact footprint. Compared to microelectromechanical system (MEMS)-based probes, the current probe configuration does not involve beam folding optics and thus allows for an overall compact probe size. These features will enable future application of SHG endomicroscopy for clinical evaluation of a wide variety of disorders where collagen architecture is altered. In particular, translation of this emerging technology into the obstetrics clinic could potentially have a dramatic impact on the understanding and prediction of preterm birth risk. It is envisioned that some parameters of the current endomicroscope originally designed for animal use (such as the probe diameter and rigid length), could be relaxed when intended for human use, offering an opportunity to further improve the imaging performance of the probe (such as a better resolution and a longer working distance). Clinical success with larger multiphoton excitation devices has recently been reported. These devices perform “optical skin biopsies” using both autofluorescence and SHG for diagnosing skin malignancies with no noted adverse effects even at an excitation power about 150 mW. This provides further optimism for the clinical potential of this reported endomicroscopy technology.

Alterations in cervical collagen structure and assembly are the earliest identified molecular changes in the cervix during pregnancy and precede measurable biomechanical changes of cervical tissue in mouse models. Progressive modification of collagen architecture is likely to be important for cervical remodeling in women as well, as women with genetic defects that alter collagen processing and assembly (e.g. Ehlers Danlos syndrome) are predisposed to PTB resulting from cervical insufficiency. A noninvasive methodology for visualizing changes in cervical collagen organization thus would have the potential to improve diagnosis and treatment in cases where preterm birth involves premature or abnormal remodeling of cervical collagen. Because the SHG signal is an intrinsic property of the assembled collagen fibrils, SHG imaging is ideally suited for visualization of collagen I at the molecular level in vivo without the need for stains or contrast agents. The endomicroscope described here has sufficient resolution to distinguish the early changes in cervical collagen architecture during normal pregnancy in mouse tissue and quantify them by image analysis, suggesting the potential clinical application of this methodology for monitoring the progress of pregnancy in women. SHG microscopy can detect abnormal cervical ripening in a mouse model of preterm birth. Application of SHG endomicroscopy technology to detect aberrations in cervical remodeling in women well in advance of premature birth onset would not only allow early and accurate prediction of PTB risk but also facilitate development of therapies for prevention. Thus, successful translation of this developing technology is likely to have a significant impact on reducing PTB rates, reducing infant mortality rates, and reducing the number of individuals who suffer from the negative lifelong health consequences of prematurity.

The engineering details of the endomicroscope include a four-quadrant piezoelectric (PZT) tube was adopted as a cantilever actuator. A double clad fiber was run through and glued to the end of the PZT tube with approximately 1 cm freely standing length as a cantilever, which was actuated by the PZT tube to perform 2-dimensional beam scanning. An open-close spiral-scanning pattern was produced when the two orthogonal pairs of electrodes on the outer surface of the PZT actuator were driven by an amplitude-modulated sine and cosine waves. Effective cantilever tip sweeping was achieved by driving the PZT actuator at or near the mechanical resonant frequency of the fiber cantilever. For a 1-cm long fiber cantilever, the resonant (spiral) scanning frequency was about 1.4 kHz, resulting in an imaging frame rate of about 2.7 frames/second with each frame consisting of 512 spirals. An approximately 500-μm scanning diameter traced by the fiber tip was achieved with a peak-to-peak drive voltage of approximately 5-140 V (depending on the fiber and how well the fiber was attached to the actuator). The corresponding beam scanning range was about 110 μm on the sample when using a compound microlens with a magnification of 0.22. The miniature scanner was housed along with the microlens into a waterproof 14-gauge ultrathin hypodermic tube with an overall outer-diameter of 2 mm and a rigid length of 32 mm.

Femtosecond pulses generated from a Ti:Sapphire laser (Chameleon Vision II from Coherent Inc.) was tuned to 890 nm with a transform-limited pulse width of approximately 150 fs. The laser has a built-in prechirp unit providing a maximum −15;000 fs2 group velocity dispersion at 890 nm, which can compensate the positive dispersion from a DCF of a length up to 50 cm. Considering the extra length of the DCF in the endomicroscope and other optical components in the beam path (such as a Faraday isolator for preventing the reflections back into the laser, the attenuator for tuning the laser power and lenses for controlling the beam diameter and coupling the beam into the DCF, etc.), which also introduce positive dispersion, a pulse stretcher based on a pair of transmission gratings (600 lines/mm) was employed to provide additional dispersion compensation. The prechirped pulses were launched into the endomicroscope probe through a coupling lens (Olympus 20×0.4 NA objective). The average incident power on the tissue sample was about 40 mW with a measured pulse width of about 450 fs after a 60-cm long DCF in the endomicroscope. The residual pulse broadening was mainly caused by the nonlinear processes in the fiber core, such as self-phase modulation. The SHG signal from the sample was collected by the microlens and then focused back into the DCF. The SHG signal, propagating mainly through the inner clad, was separated at the proximal end of the endomicroscope from the backscattered excitation light by a dichroic mirror (Semrock FF665 Di01), further filtered by a bandpass filter (Semrock FF01-445/20), and then detected by a photon ultiplier tube (PMT) (Hamamatsu H7422P). The photo current from the PMT was converted to voltage, amplified, and digitized in synchrony with the PZT actuating waveforms. Polar coordinates were chosen to represent the spiral scan leading to a (r, θ, ISHG) surface where ISHG is the signal intensity measured from the coordinate [symbol] r; θ [symbol] . For storage and analysis, the polar surface was interpolated and mapped onto a two-dimensional Cartesian surface, which was then saved as an eight-bit 1464×1464 pixel image with the range of ISHG being stretched to 256 gray levels.

Mice were housed together overnight and vaginal plugs were checked the next morning. The presence of a vaginal plug was counted as day 0.5 of the 19 day mouse gestation. On days 6, 12, 15, and 18 of gestation, mice were sacrificed and reproductive tissue was removed. Cervical and vaginal tissues were embedded in the optimal cutting temperature (OCT) compound (Tissue Tek, Indiana) and frozen in liquid nitrogen. 50 μm transverse sections were cut and mounted on glass slides. Before imaging, the entire tissue with intact epithelium or the sections were thawed at room temperature and hydrated with phosphate buffered saline. All studies were conducted in accordance with the standards of humane animal care described in the National Institutes of Health Guide for the Care and Use of Laboratory Animals, using protocols approved by an institutional animal care and research advisory committees.

Human cervical tissues from nonpregnant women were obtained at the time of hysterectomy for benign gynecological indications with no cervical pathology. Cervical tissue from a pregnant woman (36 wk) was obtained at the time of cesarean hysterectomy for indications that did not involve cervical pathology. The protocols for human tissue collection are approved by the Institutional Review Board at the University of Texas Southwestern Medical Center. Human samples were cut to include both epithelium and stroma. Tissues were frozen in OCT compound for preservation. Before imaging, the entire tissues were thawed at room temperature and hydrated with phosphate buffered saline, and then placed on the wax block with the exocervix faced outside for imaging. Trichrome Staining. Mouse cervices were collected and fixed in 4% paraformaldehyde overnight, paraffin embedded and 5-μm sections were cut and stained with Masson's Trichrome. Trichrome staining selectively stains collagen blue and smooth muscle, keratin and cytoplasm pink.

All measurements were performed on the entire field of view of each image. Fiber diameter measurements were carried out on locally normalized image sets. Briefly, images were analyzed using the FD Math function in ImageJ 1.41 k, to obtain the pixel-based intensity autocorrelation over the entire image. A 2-D Gaussian function was fit to the center of the autocorrelation image (excluding the center pixel), and 2 times the standard deviation of the mean was taken as the characteristic fiber size (n ¼ 3 animals per time point and 25-30 images per animal). Data for different days of pregnancy were compared using a one-way ANOVA followed by Tukey analysis.

Intensity measurements were carried out on globally normalized images. Day 18 images were used to define a threshold that separated SHG signal from background (i.e., the empty space on the image). Mean gray value (MGV) and fractional area of the pixels above threshold were measured using ImageJ with n ¼ 3 animals per time point and 25-30 images per animal. Data were analyzed using a one-way ANOVA followed by Tukey analysis.

The present invention can also be executed using a computer program configured to take the SHG endoscopy images of the cervical collage and determine collagen fiber morphologies using the SHG endoscopy images. Any such computer program will be fixed on a non-transitory computer readable medium. It should be noted that the computer program is programmed onto a non-transitory computer readable medium that can be read and executed by any computing device, such as a pc computer, tablet computer, phablet, smartphone, laptop, or any other suitable computing device known to or conceivable by one of skill in the art. The non-transitory computer readable medium can take any suitable form known to one of skill in the art. The non-transitory computer readable medium is understood to be any article of manufacture readable by a computer. Such non-transitory computer readable media includes, but is not limited to, magnetic media, such as floppy disk, flexible disk, hard, disk, reel-to-reel tape, cartridge tape, cassette tapes or cards, optical media such as CD-ROM, DVD, blu-ray, writable compact discs, magneto-optical media in disc, tape, or card form, and paper media such as punch cards or paper tape. Alternately, the program for executing the method and algorithms of the present invention can reside on a remote server or other networked device. Any databases associated with the present invention can be housed on a central computing device, server(s), in cloud storage, or any other suitable means known to or conceivable by one of skill in the art. All of the information associated with the programm is transmitted either wired or wirelessly over a network, via the internet, cellular telephone network, or any other suitable data transmission means known to or conceivable by one of skill in the art.

The many features and advantages of the invention are apparent from the detailed specification, and thus, it is intended by the appended claims to cover all such features and advantages of the invention which fall within the true spirit and scope of the invention. While the invention is discussed herein, with respect to the example of cervical morphology and pre-term birth, the present invention can also be used for the assessment of other pathologies. Examples of these other pathologies include but are not limited to cancer, fibrosis, and inflammation. The present invention can be used for diagnosis, monitoring of therapeutics or disease state, and guidance for intervention or surgery. The present invention is used to enable visualization of histology inside a patient in-vivo and without radio-labeling. Further, because numerous modifications and variations will readily occur to those skilled in the art, it is not desired to limit the invention to the exact construction and operation illustrated and described, and accordingly, all suitable modifications and equivalents may be resorted to, falling within the scope of the invention.

What is claimed is:

1. A method of assessing preterm birth in vivo comprising:
operating a fiber-optic scanning second harmonic generation (SHG) endoscopy imaging system;
obtaining SHG endoscopy images of cervical collagen; and
determining collagen fiber morphologies from the SHG endoscopy images.

2. The method of claim 1 further comprising identifying collagen fiber morphologies that include at least one selected from the group consisting of collagen fiber diameter, volume, porosity, and fiber bending angle.

3. The method of claim 1 further comprising using a computing device to determine the collagen fiber morphologies.

4. The method of claim 1 further comprising determining abnormal changes in collagen fiber morphologies in order to determine a risk of pre-term birth.

5. The method of claim 1 further comprising operating the SHG endoscopy imaging system in situ, in vivo, and in real time.

6. The method of claim 1 further comprising operating a SHG endoscopy system having a short pulsed laser source, a fiber optic scanning endoscope, light detection unit, and computer control.

7. The method of claim 1 further comprising obtaining images from the front and from the side using the SHG endoscopy imaging system.

8. A system for assessing preterm birth in vivo comprising:
a short pulsed laser source;
a fiber optic scanning endoscope;
a light detection unit; and
a non-transitory computer readable medium programmed with steps comprising:
guiding acquisition of SHG endoscopy images of cervical collagen;
determining collagen fiber morphologies from the SHG endoscopy images.

9. The system of claim 8 further comprising the non-transitory computer readable medium being configured for identifying collagen fiber morphologies that include at least one selected from the group consisting of collagen fiber diameter, volume, porosity, and fiber bending angle.

10. The system of claim 8 further comprising the non-transitory computer readable medium being configured for using a computing device to determine the collagen fiber morphologies.

11. The system of claim 8 further comprising the non-transitory computer readable medium being configured for determining abnormal changes in collagen fiber morphologies in order to determine a risk of pre-term birth.

12. The system of claim 8 further comprising the non-transitory computer readable medium being configured for further comprising operating the SHG endoscopy imaging system in situ, in vivo, and in real time.

13. The system of claim 8 further comprising the non-transitory computer readable medium being configured for further comprising obtaining images from the front and from the side using the SHG endoscopy imaging system.

14. The system of claim 8 further comprising the non-transitory computer readable medium being configured for determining structural aberrations in collagen fibers of the cervical collagen.

15. The system of claim 8 further comprising the non-transitory computer readable medium being configured for calculating a risk for preterm birth from the collagen fiber morphology.

16. The system of claim 8 further comprising the non-transitory computer readable medium being configured for locally normalizing the SHG endoscopy images by contrast stretching each image individually to fill and 8-bit grayscale.

17. A system for assessment of a pathology comprising:
a short pulsed laser source;
a fiber optic scanning endoscope;
a light detection unit; and
a non-transitory computer readable medium programmed with steps comprising:
guiding acquisition of SHG endoscopy images of the pathology;
determining morphology of the pathology from the SHG endoscopy images.

18. A method for assessment of a pathology comprising:
operating a fiber-optic scanning second harmonic generation (SHG) endoscopy imaging system;
obtaining SHG endoscopy images of the pathology; and
determining morphology for the pathology from the SHG endoscopy images.
